(19) United States
(12) Patent Application Publication
(10) Pub. No.: US 2025/0268962 A1
(43) Pub. Date: Aug. 28, 2025

(54) COMPOSITION FOR INFLAMMATORY BOWEL DISEASE

(71) Applicant: SONE FARM CO., LTD., Tokyo (JP)

(72) Inventor: Masanori Sugiyama, Hiroshima-shi (JP)

(73) Assignee: SONE FARM CO., LTD., Tokyo (JP)

(21) Appl. No.: 19/200,257

(22) Filed: May 6, 2025

Related U.S. Application Data
(63) Continuation of application No. 17/794,926, filed on Jul. 22, 2022.

(30) Foreign Application Priority Data
Oct. 19, 2020 (JP) 2020-175473

(51) Int. Cl. A61K 35/747; A23L 29/00; A23L 33/135; A61K 31/715; A61P 1/04

(52) U.S. Cl. CPC A61K 35/747; A23L 29/065; A23L 33/135; A61K 31/715; A61P 1/04

(57) ABSTRACT

A composition comprising, as an active ingredient, a cell body of a fig-derived lactic acid bacterium belonging to Lactobacillus paracasei, a cultured or fermented product thereof, or a polysaccharide produced thereby is effective in improving, preventing or treating an inflammatory bowel disease and can be particularly used as a food or drink, a medicament or a feed for improving, preventing or treating ulcerative colitis or Crohn's disease.

### TECHNICAL FIELD

The present invention relates to a composition for an inflammatory bowel disease. More specifically, the present invention relates to a composition for improving, preventing or treating an inflammatory bowel disease, comprising, as an active ingredient, a cell body of a fig-derived lactic acid bacterium belonging to Lactobacillus paracasei, a cultured or fermented product thereof, or a polysaccharide produced thereby.

### BACKGROUND ART

In recent years, the number of patients with an inflammatory bowel disease (IBD) has been increasing, as if it were linked to changes in eating habits. An inflammatory bowel disease is a disease in which as a result of abnormalities in human immune system, self-immune cells attack intestinal cells to cause inflammation in the intestine. Symptoms include chronic diarrhea, bloody stools, and abdominal pain. According to a survey in Japan in 2020, young people (20 to 30 year-old) have the highest incidence rate, and an inflammatory bowel disease is especially designated as an intractable disease. An inflammatory bowel disease can be broadly divided into ulcerative colitis (UC) and Crohn's disease (CD).

Ulcerative colitis causes inflammation of the large intestine, resulting in diarrhea and bloody stools. Typical symptoms include diarrhea of about 5 to 10 times per day and also continuous bloody stools. The lesion area and severity differ from patient to patient, and there are cases of from mild cases in which mild inflammation is caused only in rectum and in which a slight amount of blood is adhered to stools, to severe cases in which strong inflammation is cased in the entire large intestine and in which diarrhea and bloody stools are caused 20 or more times per day, with anemia, abdominal pain, fever, etc. In many patients, proper treatment improves symptoms, but discontinuation of treatment may cause symptoms to recur.

Crohn's disease may cause inflammation in the entire gastrointestinal tract of from mouth to anus. The lesion sites are mainly small intestine, large intestine, and anus.

Symptoms of diarrhea and abdominal pain, fever and weight loss are often observed, and anus may be swollen and painful. Crohn's disease forms deep ulcers in intestines, which may narrow or puncture the intestines.

As mentioned above, an inflammatory bowel disease such as ulcerative colitis or Crohn's disease is considered to be an autoimmune disease triggered by abnormalities in human immune system, and the patient's own immune cells attack the intestinal cells, resulting in that chronic inflammation of the intestinal tract occurs. The patients suffer from chronic diarrhea and abdominal pain, but it is difficult to cure them with current medical techniques once they occur, and the treatment must be continued for a lifetime, so that effective treatments and drugs are desired to be developed as soon as possible.

The current treatment for ulcerative colitis prescribes a 5-aminosalicylic acid formulation when inflammation is mild and when symptoms are not severe. This drug acts directly on inflamed large intestine mucosa to exert its effect. Therefore, in addition to oral administration, there is also a method of injecting it through anus as a suppository or enema. On the other hand, for severely ill patients, steroids that suppress abnormal autoimmune reactions are prescribed. In cases where steroids are ineffective or where relapse occurs when the dose of steroids is reduced or when the administration is discontinued, strong immunosuppressants or biologics made from biological proteins are injected. In addition, a treatment called blood cell component removal therapy, in which a part of blood is circulated outside the body to remove immune cells from the blood, is also performed.

On the other hand, recent research advances have led to the belief that the inflammation of an inflammatory bowel disease is caused by gut microbiota. Since probiotics such as lactic acid bacteria and bifido bacteria prevent and improve enteritis, the probiotics are attracting attention as one of the treatment methods for an inflammatory bowel disease, and many reports have been made on the treatments of an inflammatory bowel disease with probiotics (Non-Patent Document 1).

In addition, Patent Document 1 discloses that lactic acid bacteria such as Lactobacillus plantarum, Lactobacillus rhamnosus, Lactobacillus fermentum, and Lactobacillus paracasei are used in the treatment of an inflammatory bowel disease. Patent Document 2 discloses that lactic acid bacteria of Lactobacillus buchneri have an anti-inflammatory effect on an inflammatory bowel disease. Patent Document 3 discloses that lactic acid bacteria of Lactobacillus gasseri lactic acid bacteria are used in the alleviation of an inflammatory bowel disease.

### PRIOR ART DOCUMENTS

### Patent Documents

Patent Document 1: WO 2007/040446 A

Patent Document 2: JP 2010-99024 A

Patent Document 3: JP 2014-113137 A

### Non-Patent Documents

Non-Patent Document 1: Journal of Intestinal Microbiology, Vol. 23, No. 3, 2009, 193-201

### SUMMARY OF INVENTION

### Problem to be Solved by Invention

Under such background arts, it has been desired to develop a new composition for improving, preventing or treating an inflammatory bowel disease, containing a lactic acid bacterium as an active ingredient. Thus, the problem to be solved by the present invention is to provide a new composition effective in improving, preventing or treating an inflammatory bowel disease, comprising a lactic acid bacterium as an active ingredient.

### Means for Solving the Problem

The present inventor has made conducted intensive studies with the aim of developing a new composition for improving, preventing or treating an inflammatory bowel disease. As a result, the present inventor has found that a fig-derived lactic acid bacteria belonging to Lactobacillus paracasei and polysaccharides produced thereby exhibit an action that is effective in improving, preventing or treating an inflammatory bowel disease including ulcerative colitis and Crohn's disease. On the basis of these findings, the present inventor has further studied and completed the present invention.

In one aspect of the present invention, the present invention relates to a composition for improving, preventing or treating an inflammatory bowel disease, comprising, as an active ingredient, a cell body of a fig-derived lactic acid bacterium belonging to Lactobacillus paracasei, a cultured or fermented product thereof, or a polysaccharide produced thereby.

The composition of the present invention can be preferably used to improve, prevent or treat ulcerative colitis and Crohn's disease.

In the composition of the present invention, the lactic acid bacterium is a fig-derived lactic acid bacterium belonging to Lactobacillus paracasei and particularly preferably Lactobacillus paracasei strain IJH-SONE68 (Accession No. NITE BP-02242) or a lactic acid bacterium equivalent thereto.

In the composition of the present invention, the polysaccharide produced by the lactic acid bacterium is preferably an acidic polysaccharide or a neutral polysaccharide. The acidic polysaccharide preferably includes an acidic polysaccharide composed mainly of glucoses and mannoses, and the neutral polysaccharide preferably includes a neutral polysaccharide having a structure in which N-acetylglucosamines are linked with each other via α-1,6 bond.

The composition of the present invention is preferably a food or drink composition, and the food or drink includes a beverage, a functional food, a fermented food, and a supplement.

In addition, the composition of the present invention is preferably a pharmaceutical composition. Furthermore, the composition of the present invention is preferably a feed composition.

### Effect of Invention

The composition of the present invention is effective in improving, preventing or treating an inflammatory bowel disease and can be particularly used to improve, prevent or treat ulcerative colitis and Crohn's disease. The composition of the present invention is preferably a food or drink composition for improving, preventing or treating an inflammatory bowel disease, and the food or drink is preferably a beverage, a functional food, a fermented food, or a supplement. In addition, the composition of the present invention is preferably a pharmaceutical composition, and a feed composition is also preferable. Since the composition of the present invention comprises, as an active ingredient, a cell body of a fig-derived lactic acid bacterium belonging to Lactobacillus paracasei, a cultured or fermented product thereof, or a polysaccharide produced thereby, the composition of the present invention has high safety, can be applied for a long period of time, can be supplied in a large amount at low cost, and has extremely high utility and practicality.

### BRIEF DESCRIPTION OF DRAWINGS

FIG. 1 illustrates an isolation profile of polysaccharides of Lactobacillus paracasei strain IJH-SONE68 with anion exchange chromatography (TOYOPEARL DEAE-650M resin (Tosoh Corporation)). The polysaccharides were eluted with NaCl having a gradient concentration of 0 mM to 500 mM (broken line), and the polysaccharides in each fraction were monitored at 490 nm by a phenol sulfuric acid method (straight line).

FIG. 2 illustrates each NMR profile obtained by subjecting a neutral polysaccharide, which was obtained by purifying polysaccharides of Lactobacillus paracasei strain IJH-SONE68 with anion exchange column chromatography, to proton-NMR and carbon-NMR. (A) in FIG. 2 is the NMR profile of proton-NMR, and (B) in FIG. 2 is the NMR profile of carbon-NMR.

FIG. 3 illustrates results of structurally analyzing a neutral polysaccharide on the basis of the NMR profiles. These structural analysis results revealed that the neutral polysaccharide of Lactobacillus paracasei strain IJH-SONE68 has a structure in which N-acetylglucosamines are linked with each other via α-1,6 bond.

FIG. 4 illustrates the inhibition rate of a culture liquid supernatant of Lactobacillus paracasei strain IJH-SONE68 and polysaccharides against the production of interleukin-8 (IL-8) in Caco-2 cells that are intestinal epithelial cell models.

FIG. 5 illustrates the inhibition rate of a culture liquid supernatant of Lactobacillus paracasei strain IJH-SONE68 against the production of tumor necrosis factor-α (TNF-α) and interleukin-6 (IL-6) in mouse macrophage cultured cells RAW264.7.

FIG. 6 illustrates an improvement effect of polysaccharides produced by Lactobacillus paracasei strain IJH-SONE68 on diarrhea of ulcerative colitis model mice.

FIG. 7 illustrates an improvement effect of polysaccharides produced by Lactobacillus paracasei strain IJH-SONE68 on bloody stools of ulcerative colitis model mice.

FIG. 8 illustrates an improvement effect of polysaccharides produced by Lactobacillus paracasei strain IJH-SONE68 on diarrhea and bloody stools of ulcerative colitis model mice (the total value of the diarrhea score and the bloody stools score (Disease Activity Index (DAI value)).

FIG. 9 illustrates an influence of polysaccharides produced by Lactobacillus paracasei strain IJH-SONE68 on the length of the large intestine of ulcerative colitis model mice.

FIG. 10 illustrates an improvement effect of neutral and acidic polysaccharides produced by Lactobacillus paracasei strain IJH-SONE68 on diarrhea of ulcerative colitis model mice.

FIG. 11 illustrates an improvement effect of neutral and acidic polysaccharides produced by Lactobacillus paracasei strain IJH-SONE68 on bloody stools of ulcerative colitis model mice.

FIG. 12 illustrates an improvement effect of neutral and acidic polysaccharides produced by Lactobacillus paracasei strain IJH-SONE68 on diarrhea and bloody stools of ulcerative colitis model mice (the total value of the diarrhea score and the bloody stools score (Disease Activity Index (DAI value)).

FIG. 13 illustrates an influence of neutral and acidic polysaccharides produced by Lactobacillus paracasei strain IJH-SONE68 on the length of the large intestine of ulcerative colitis model mice.

FIG. 14 illustrates an influence of neutral and acidic polysaccharides produced by Lactobacillus paracasei strain IJH-SONE68 on myeloperoxidase (MPO) activity of the large intestine of ulcerative colitis model mice.

### EMBODIMENTS FOR CARRYING OUT INVENTION

The following detailed disclosures are made on the composition provided by the present invention for in improving, preventing or treating an inflammatory bowel disease, comprising, as an active ingredient, a cell body of a fig-derived lactic acid bacterium belonging to Lactobacillus paracasei, a cultured or fermented product thereof, or a polysaccharide produced thereby.

### 1. The Lactic Acid Bacterium Used in the Present Invention

The lactic acid bacterium used in the present invention is a fig-derived lactic acid bacterium belonging to Lactobacillus paracasei. Specifically, the lactic acid bacterium preferably includes Lactobacillus paracasei strain IJH-SONE68 that was isolated and identified from leaves of a fig according to the present invention. This strain was nationally deposited under the accession number of NITE P-02242 at Patent Microorganisms Depositary, National Institute of Technology and Evaluation (#122, 2-5-8 Kazusakamatari, Kisarazu-shi, Chiba 292-0818, Japan) on Apr. 19, 2016. The deposition was then transferred to an international deposit under the Budapest Treaty and given the international deposit accession number of NITE BP-02242 on May 26, 2017. In addition, this strain is disclosed in WO 2018/225556 A, WO 2018/225557 A, WO 2019/208149 A, WO 2019/208150 A, WO 2019/208151 A, and the like. Lactobacillus paracasei strain IJH-SONE68 is a catalase-negative, gram-positive bacillus, and has mycological properties of forming a white colony and the mycological characteristic of conditional heterolactic fermentation. Furthermore, the strain has an ability to produce polysaccharides.

A lactic acid bacterium equivalent to Lactobacillus paracasei strain IJH-SONE68 can also be used in the present invention. Here, the equivalent lactic acid bacterium indicates a lactic acid bacterium that belongs to Lactobacillus paracasei and has an action of improving, preventing or treating an inflammatory bowel disease, like Lactobacillus paracasei strain IJH-SONE68. In addition, the equivalent lactic acid bacterium also indicates a lactic acid bacterium that produces polysaccharides having an action of in improving, preventing or treating an inflammatory bowel disease, like the polysaccharides produced by Lactobacillus paracasei strain IJH-SONE68. These equivalent lactic acid bacteria are obtained, for example, by performing usual mutation treatment technique, such as mutation and genetic recombination, on Lactobacillus paracasei strain IJH-SONE68 and, in addition, may be bacterial strains that have been bred by selecting natural mutation strains of Lactobacillus paracasei strain IJH-SONE68, and the like.

2. The active ingredient in the present invention

The composition of the present invention contains, as an active ingredient, a cell body of the above-mentioned lactic acid bacterium, a cultured or fermented product thereof, or a polysaccharide produced thereby.

The lactic acid bacterium can be cultured by a commonly used culture method such as liquid stationary culture, using a commonly used MRS medium or a modified medium thereof. The lactic acid bacterium can promote its growth by the culture in the presence of a fruit juice of a pineapple genus plant or its extract (WO 2011/046194 A). In addition, the lactic acid bacterium can also promote its growth by the culture in the presence of sake lees, sake lees extract or sake lees enzymes (JPH 03-172171 A, JPH 05-15366 A, and JP 2835548 B). As the lactic acid bacterium, a culture obtained after the cultivation may be used as it is, a supernatant of the obtained culture liquid may be used, the obtained culture liquid may be diluted or concentrated to be used, or the cell body recovered from the culture may be used. In addition, as long as the effect of the present invention is not impaired, various additional operations such as heat and freeze-dry may also be performed after the cultivation. The lactic acid bacterium of the present invention may be viable or dead and may include both viable and dead bacteria, while polysaccharides are adhered to the cell surface of the lactic acid bacterium. The dead lactic acid bacterium may be crushed and preferably has polysaccharides adhered to the cell surface thereof. In addition, the fermented product of the lactic acid bacterium can be obtained by fermenting the lactic acid bacterium usually using glucose or the like as a nutrient source, and further using yeast extract, fruit juice of pineapple genus plants, sake lees, distilled spirit lees, etc., if necessary.

The polysaccharides produced by the lactic acid bacterium can be obtained by isolating and purifying them from a culture of fig-derived lactic acid bacteria belonging to Lactobacillus paracasei according to an ordinary method. Specifically, for example, the polysaccharides can be obtained by removing the cell body from a culture of fig-derived lactic acid bacteria belonging to Lactobacillus paracasei by centrifugation, and precipitating polysaccharides from the obtained culture using ethanol, acetone, or the like. In addition, the polysaccharides can be obtained by further isolating and purifying them by ion exchange chromatography.

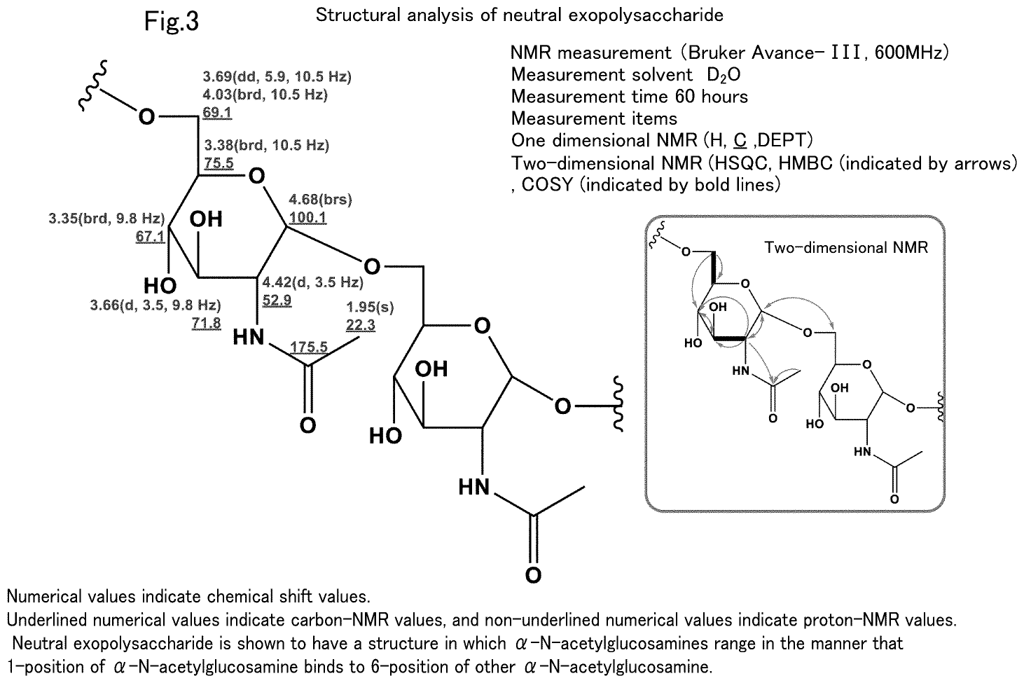

In the present invention, the polysaccharides produced by the lactic acid bacterium specifically include a neutral polysaccharide having a structure in which N-acetylglucosamines are linked with each other via α-1,6 bond, and an acidic polysaccharide composed mainly of glucoses and mannoses. This neutral polysaccharide can be obtained by isolating and purifying the polysaccharides obtained from a culture of Lactobacillus paracasei strain IJH-SONE68 by anion exchange chromatography, as disclosed in Example 2 herein below. It was revealed from the proton-NMR and carbon-NMR profiles illustrated in FIG. 3 that this neutral polysaccharide has a structure in which N-acetylglucosamines are linked with each other by α-1,6 bonds. In addition, the Lactobacillus paracasei strain IJH-SONE68 secretes an acidic polysaccharide mainly composed of glucose and mannose outside the cell body. More specifically, this acidic polysaccharide is composed of glucose, mannose, galactose, and rhamnose, and their composition ratio is approximately 10:170: 2:1.

### 3. The Composition in the Present Invention

The composition of the present invention can be used in various forms of a food or drink composition, a pharmaceutical composition, and a feed composition.

The food or drink of the food or drink composition are not particularly limited but include beverages such as soft drinks, carbonated drinks, nutritional drinks, fruit juice beverages, and lactic acid bacteria beverages, concentrated stock solutions of these beverages, powders for the preparation of these beverages, and the like; ice cream, sherbet and ice confectionery such as shaved ice; confectioneries such as candy, gummy, cereal, chewing gum, candy, gum, chocolate, tablet candy, snack, biscuit, jelly, jam, cream, and baked confectionery; dairy products such as processed milk, milk drink, fermented milk, drink yogurt, and butter; bread; enteral nutritious food, liquid food, childcare milk, sports drink; food such as puree; seat sake; and other functional foods. The food or drink may be supplements, and the supplements may be in the form of, for example, granules, powders, or tablets. In addition, the food or drink may be a fermented food that can be obtained by fermenting various food or drink with the lactic acid bacteria.

The food or drink as disclosed above may be prepared by adding a cell body of the lactic acid bacterium, a cultured or fermented product thereof, or a polysaccharide produced thereby, to raw materials of food or drink, or prepared in the same manner as a usual food or drink. The addition of a cell body of the lactic acid bacterium, a cultured or fermented product thereof, or a polysaccharide produced thereby may be performed at any stage of the process of preparing the food or drink. The food or drink may be prepared after a fermentation process of the added lactic acid bacteria. Examples of such food or drink include fermented foods such as lactic acid bacterium beverages and fermented milks, and the like.

The content of a cell body of the lactic acid bacterium or a cultured or fermented product thereof in the food or drink composition may be appropriately determined depending on the embodiment of the food or drink, but is usually the one so that a cell body of the lactic acid bacterium is usually contained in the food or drink composition preferably in the range of 1×10⁶ to 1×10¹² cfu/g or 1×10⁶ to 1×10¹² cfu/ml, more preferably in the range of 1×10⁷ to 1×10¹¹ cfu/g or 1×10⁷ to 1×10¹¹ cfu/ml. In the case where the lactic acid bacterium is a dead cell body, the cfu/g or cfu/ml can be replaced with the number of cells per g or the number of cells per ml. In the case of the polysaccharide produced by the lactic acid bacterium, the polysaccharide is usually contained in the food or drink composition at an amount of 0.001% or more by weight, preferably at an amount of 0.01% or more by weight, in terms of the weight of the polysaccharide.

The pharmaceutical composition is usually used by blending a cell body of the lactic acid bacterium, a cultured or fermented product thereof or a polysaccharide produced thereby with a physiologically acceptable liquid or solid of a pharmaceutical carrier usually used, followed by the formulation. The dosage form of the pharmaceutical composition is not particularly limited, but includes tablets, pills, powders, solutions, suspensions, emulsions, granules, capsules, syrups, suppositories, injections, ointments, patches, eye drops, and nose drops.

The content of a cell body of the lactic acid bacterium or a cultured or fermented product thereof in the pharmaceutical composition may be appropriately determined depending on the dosage form, the dosage regimen, the age and sex of a subject, the kind of disease, the degree of disease, other conditions and the like, but is usually the one so that a cell body of the lactic acid bacterium is usually contained in the pharmaceutical composition preferably in the range of 1×10⁶ to 1×10¹² cfu/g or 1×10⁶ to 1×10¹² cfu/ml, more preferably in the range of 1×10⁷ to 1×10¹¹ cfu/g or 1×10⁷ to 1×10¹¹ cfu/ml. In the case where the lactic acid bacterium is a dead cell body, the cfu/g or cfu/ml can be replaced with the number of cells per g or the number of cells per ml. In the case of the polysaccharide produced by the lactic acid bacterium, the polysaccharide is usually contained in the pharmaceutical composition at an amount of 0.001% or more by weight, preferably at an amount of 0.01% or more by weight, in terms of the weight of the polysaccharide.

The administration timing of the pharmaceutical composition of the present invention is not particularly limited but may be appropriately chosen depending on a subject to be applied. The pharmaceutical composition may also be administered prophylactically or used in a maintenance therapy. The administration mode may be preferably appropriately determined depending on the dosage form, age, sex and other conditions of the administered subject, the degree of symptoms of the administered subject, and the like. In any case, the pharmaceutical composition of the present invention may be administered once per day, administered dividedly into two or more times or administered once every several days or weeks.

Examples of the feed of a feed composition include pet food, livestock feed and fish feed. Such a feed may be prepared by mixing common feed, for example, cereals, cakes, brans, fish meals, bone meals, oils and fats, skim milk powders, wheys, bitterns, mineral feeds, yeasts, or the like with a cell body of the lactic acid bacterium, a cultured or fermented product thereof or a polysaccharide produced thereby. In addition, for example, like the case of silage, a feed may be prepared through a fermentation process with the lactic acid bacterium added thereto. The prepared feed may be orally administered to general mammals, livestock, farmed fishes, pet animals or the like. In the case of farmed fishes, it may be adopted to spread fermented products, to which a cell body of the lactic acid bacterium, a cultured or fermented product thereof or a polysaccharide produced thereby has been added, to the farmed place of fishes.

The content of a cell body of the lactic acid bacterium or a cultured or fermented product thereof in the feed composition may be appropriately determined depending on the embodiment of the feed or the administered subject, but is usually the one so that a cell body of the lactic acid bacterium is usually contained in the feed composition preferably in the range of 1×10⁶ to 1×10¹² cfu/g or 1×10⁶ to 1×10¹² cfu/ml, more preferably in the range of 1×10⁷ to 1×10¹¹ cfu/g or 1×10⁷ to 1×10¹¹ cfu/ml. In the case where the lactic acid bacterium is a dead cell body, the cfu/g or cfu/ml can be replaced with the number of cells per g or the number of cells per ml. In the case of the polysaccharide produced by the lactic acid bacterium, the polysaccharide is usually contained in the feed composition at an amount of 0.001% or more by weight, preferably at an amount of 0.01% or more by weight, in terms of the weight of the polysaccharide.

### 4. Use of the Composition of the Present Invention

A cell body of a fig-derived lactic acid bacterium belonging to Lactobacillus paracasei, a cultured or fermented product thereof, or a polysaccharide produced thereby has an action of improving, preventing or treating an inflammatory bowel disease including ulcerative colitis and Crohn's disease. Therefore, a composition containing, as an active ingredient, a cell body of a fig-derived lactic acid bacterium belonging to Lactobacillus paracasei, a cultured or fermented product thereof, or a polysaccharide produced thereby can be used to improve, prevent or treat an inflammatory bowel disease including ulcerative colitis and Crohn's disease.

More specifically, as shown on Example 3 disclosed herein after, it has been revealed that when the culture liquid supernatant of Lactobacillus paracasei IJH-SONE68 (Accession No. NITE BP-02242) or polysaccharides thereof is added to human colon cancer-derived Caco-2 cells, which are intestinal epithelial model cells and in which inflammation has been induced by Salmonella and Campylobacter, the expression and secretion of interleukin-8 (IL-8), which is one of inflammatory cytokines, are suppressed.

In addition, it has been revealed that when the culture liquid supernatant of Lactobacillus paracasei IJH-SONE68 (Accession No. NITE BP-02242) is added to cultured mouse macrophage cells RAW264.7 in which inflammation has been induced by lipopolysaccharide, the expression and secretion of tumor necrosis factor-α (TNF-α) and interleukin-6 (IL-6), which are inflammatory cytokines, are suppressed.

Furthermore, it has been revealed that when polysaccharides, neutral polysaccharides, acidic polysaccharides, or the like of Lactobacillus paracasei IJH-SONE68 (Accession No. NITE BP-02242) are orally administered to ulcerative colitis model mice which have been developed by ingestion of sodium dextran sulfate (DSS), diarrhea and bloody stools are suppressed, and myeloperoxidase (MPO) activity in the large intestine, which is an indicator of inflammation intensity, is suppressed, whereby the worsening of the disease condition is suppressed.

In view of the foregoing, it has been revealed that a cell body of a fig-derived lactic acid bacterium belonging to Lactobacillus paracasei, a cultured or fermented product thereof, or a polysaccharide produced thereby is effective in improving, preventing or treating an inflammatory bowel disease including ulcerative colitis and Crohn's disease.

In addition, since the composition of the present invention is effective in improving, preventing or treating an inflammatory bowel disease, it can be particularly used as a material for foods or drinks used for maintaining and promoting health. In addition, since the improvement, prevention or treatment of an inflammatory bowel disease leads to the maintenance, promotion, recovery, and the like of physical strength, the composition of the present invention is also particularly used as a material for foods or drinks used for the maintenance, promotion, and recovery of physical strength.

### EXAMPLES

Hereinafter, the present invention will be disclosed in more detail with reference to examples, but the present invention is not limited by these examples.

### Example 1

### Isolation and Identification of Lactic Acid Bacterium

### 1. Isolation of Lactic Acid Bacterium Sample

The leaves, stems, and fruits of a fig (variety “TOYOMITSU HIME”) were chosen and cut into pieces of 2 to 3 mm using sterilized tweezers and scissors. Every five to six pieces were each then placed in a sterilized test tube containing MRS liquid medium, and statically cultured at 28° C. and 37° C. until the MRS medium as a standard medium for a lactic acid bacterium became turbid (proliferated). By the way, it took 2to 4 days for the proliferation of the lactic acid bacterium candidate strains to be visible.

A part of each culture liquid of the lactic acid bacterium candidate strains was subjected to a line drawing paint on MRS agar medium using a disposable loop, followed by stationary culture. Among colonies formed on the agar medium, all of differently colored, lustrous and shaped colonies were picked up and subjected to a line drawing paint on a fresh MRS agar medium, and the colonies were purified.

H₂O₂ test was performed for each purified colony to verify the presence or absence of the production of a catalase enzyme. This is a test method for observing the presence or absence of oxygen generated when catalase is present, which is observed when microbial bodies are exposed to 10% H₂O₂ solution. By the way, a lactic acid bacterium produces no catalase. As a result of attempting the search and isolation from a fig, one lactic acid bacterium candidate strain showing catalase-negative was obtained from the leaves of a fig as the isolation source.

### 2. Identification of the Isolated Strain

The aforementioned lactic acid bacterium candidate strain was again cultured in MRS liquid medium, and the microbial bodies were obtained by centrifugation. After the microbial bodies were treated with cell wall lytic enzyme, a genomic DNA was extracted using DNAzol reagent.

According to the method as disclosed in Lane, DJ (1991), “16S/23S rRNA sequencing”, Nucleic Acid Techniques in Bacterial Systematics, pp. 115-175, edited by E. Stackebrandt & M. Goodfellow. Chichester: Wiley, a genomic DNA PCR was performed using a genomic DNA as a template and using 27f primer and 1525r primer, thereby to amplify 16S rDNA part. Then, an objective fragment was recovered from agarose gel according to NucleoSpin Gel and PCR Clean-up kit (manufactured by Mahalay Nagel). A sequencing reaction by a dye terminator method for sequencing a base sequence was performed with Big Dye Terminator Cycle Sequencing FS Ready Reaction Kit ver. 3.1 (manufactured by ThermoFisher Scientific), and analysis was made with ABI PRISM 3130xl Genetic Analyzer (manufactured by ThermoFisher Scientific). The base sequence of the analyzed 16S rDNA was subjected to a homology search by BLAST program and compared with the database of DNA data bank (DDBJ/EMBL/GenBank) to make a taxonomic identification on the isolated strain.

The lactic acid bacterium candidate strain isolated from leaves of a fig was named strain IJH-SONE68 and identified as Lactobacillus paracasei because it was 100% identical to a base sequence which was in the strain of Lactobacillus paracasei R094 already registered in DNA data bank (DDBJ/EMBL/GenBank) and which had NR-025880 as the accession number of the base sequence.

This strain was nationally deposited under the accession number of NITE P-02242 at Patent Microorganisms Depositary, National Institute of Technology and Evaluation (#122, 2-5-8 Kazusakamatari, Kisarazu-shi, Chiba 292-0818, Japan) on Apr. 19, 2016. The deposition was then transferred to an international deposit under the Budapest Treaty and given the international deposit accession number of NITE BP-02242 on May 26, 2017.

### 3. Mycological Properties of Separated and Identified Lactic Acid Bacterium

The aforementioned isolated and identified lactic acid bacterium strain IJH-SONE68 was a catalase-negative, gram-positive rod and had a white colony forming property, and further had the characteristic of conditional hetero-lactic acid fermentation and the ability of producing polysaccharides.

### 4. Saccharide Assimilation Ability of the Isolated and Identified Lactic Acid Bacterium

(1). Test method of Assimilation Ability

The strain IJH-SONE68 was investigated for the assimilation ability of 49 kinds of saccharides according to the following test method.

The strain IJH-SONE68 was statically cultured in MRS liquid medium until the proliferation stationary phase. The microbial bodies obtained by centrifugation were washed with an appropriate amount of a suspension medium (manufactured by BioMeieux), and finally suspended in 2 mL of a suspension medium. A portion of the resultant suspension was added to 5 mL of a suspension medium to determine an amount (n) for McFarland turbidity to become 2. Subsequently, 2n of a microbial solution was added to API 50 CHL medium (manufactured by BioMerieux), and this solution was dispended to each well of API 50 CHL kit (manufactured by BioMerieux, 49 kinds of saccharides were coated on the bottom of each well). Finally, mineral oil was overlaid and set in a tray containing a sterilized water. After culturing at 37° C. for 48 hours, the presence or absence of the assimilation ability was assessed by observing the change in color tone in each well.

### (2). Test Results of the Assimilation Ability

Table 1 shows the results of investigating the assimilation ability of the strain IJH-SONE68 against 49 kinds of saccharides.

TABLE 1
Assimilation abilities of the strain IJH-SONE68
against saccharides

| Substrates | Assimilation abilities |
|---|---|
| Control | − |
| Glycerol | − |
| Erythritol | − |
| D-Arabinose | − |
| L-Arabinose | − |
| D-Ribose | + |
| D-Xylose | − |
| L-Xylose | − |
| D-Adonitol | + |
| Methyl-βD-xylopyranoside | − |
| D-Galactose | + |
| D-Glucose | + |
| D-Fructose | + |
| D-Mannose | + |
| L-Sorbose | + |
| L-Rhamnose | − |
| Dulcitol | − |
| Inositol | − |
| D-Mannitol | + |
| D-Sorbitol | − |
| Methyl-αD-mannopyranoside | − |
| Methyl-αD-glucopyranoside | − |
| N-Acetylglucosamine | + |
| Amygdalin | − |
| Arbutin | − |
| Esculin + Ferric citrate | + |
| Salicin | + |
| D-Cellobiose | + |
| D-Maltose | + |
| D-Lactose | − |
| D-Melibiose | − |
| D-Sucrose | + |
| D-Trehalose | + |
| Inulin | − |
| D-Melezitose | + |
| D-Raffinose | − |
| Starch | − |
| Glycogen | − |
| Xylitol | − |
| Gentibiose | + |
| D-Turranose | − |
| D-Lyxose | − |
| D-Tagatose | + |
| D-Fucose | − |
| L-Fucose | − |
| D-Arabitol | − |
| L-Arabitol | − |
| Gluconic acid | + |
| 2-Ketogluconic acid | − |
| 5-Ketogluconic acid | − |

In Table 1, + indicates the possession of assimilation ability, and − indicates no possession of assimilation ability.

### Example 2

### 1. Isolation and Purification of Polysaccharides Produced by the Strain IJH-SONE68

Polysaccharides produced by the strain IJH-SONE68 were isolated and purified according to the following method.

The strain IJH-SONE68 was statically cultured in MRS liquid medium until the proliferation stationary phase. 5 mL of the resultant culture solution was used as a seed culture solution, and inoculated on 5 L of a semisynthetic medium for producing exopolysaccharides (the composition thereof will be disclosed herein below), followed by static culture at 37° C. for 120 hours. After the resultant culture solution was cooled to 4° C., proteins contained in the culture supernatant were denatured, and 202.5 mL of a 100% trichloroacetic acid aqueous solution was added thereto, mixed and allowed to stand for 30 minutes to remove them as precipitates in a later step. After the precipitates were removed by centrifugation, an equal amount of acetone was added to the collected supernatant and mixed, and the resultant mixture was allowed to stand at 4° C. overnight to precipitate polysaccharides produced by the strain IJH-SONE68. The precipitates were collected by centrifugation, and the resultant precipitates were then washed with 250 mL of 70% ethanol. After the precipitates were air-dried, 75 mL of 50 mM Tris-HCl buffer (pH 8.0) was added to the resultant precipitates and mixed for 1 hour to dissolve the precipitates. After insoluble impurities were removed by centrifugation to recover a supernatant, 750 μL of 1 mg/mL DNase solution (Worthington, Inc.) and 750 μL of 1 mg/mL RNase solution (Nacalai Tesque, Inc.) were each added to the recovered supernatant, followed by being allowed to react at 37° C. for 8 hours. Subsequently, 750 μL of 2 mg/mL proteinase K solution (manufactured by Wako Pure Chemical Industries, Ltd.) was added, and the resultant mixture was reacted at 37° C. for 16 hours. The resultant solution after the reaction was cooled to 4° C., the added enzymes were each denatured, and 8.75 mL of a 100% trichloroacetic acid aqueous solution was then added thereto, mixed and allowed to stand for 1 hour to remove the enzymes as precipitates in the next centrifugation. The resultant precipitates were removed by centrifugation to obtain a supernatant, 262.5 mL of 100% ethanol was added to the obtained supernatant, the resultant mixture was thoroughly mixed, and the polysaccharides produced by the strain IJH-SONE68 strain were then recovered as precipitates by centrifugation. After the precipitates were washed with 50 mL of 70% ethanol, the precipitates were air-dried, an appropriate amount (about 25 mL) of a purified water was added thereto, and the resultant mixture was allowed to stand overnight at 4° C. to dissolve the polysaccharides. For the polysaccharides sample after the dissolution, small molecules such as monosaccharides in the recovered sample were removed using an ultrafiltration unit (Merck Ltd.) of 10,000 MWCO while replacing the solvent with a purified water, and a purified polysaccharide sample was thus obtained.

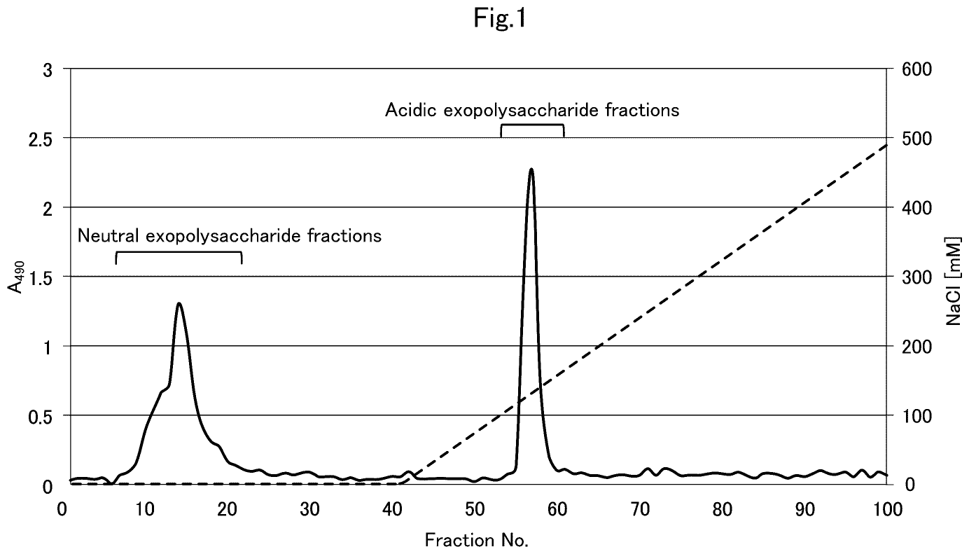

The purified polysaccharide sample was applied to an open column (2.5×22 cm) packed with TOYOPEARL DEAE-650M resin (Tosoh Corporation) previously equilibrated with 50 mM Tris-HCl buffer (pH 8.0), and column work was performed to isolate and purify the sample to neutral polysaccharide fractions and acidic polysaccharide fractions. The same buffer was used as an elution solution, and a flow rate was fixed at 1 mL/min. In addition, eluates were collected in different test tubes at every 6 mL. First, from the beginning to 240 minutes, elution was made with the same buffer (Test Tube Nos. 1 to 40). Next, from 240 minutes to 600 minutes, a concentration gradient of 0 to 500 mM NaCl was prepared using the same buffer, and elution was continued with the gradient (Test Tube Nos. 41 to 100). The column isolation spectrum is illustrated in FIG. 1. After the presence of polysaccharides was confirmed by a phenol sulfuric acid method (disclosed below) for all the samples eluted in the test tubes, the confirmed solutions in the test tubes were collected as neutral polysaccharide fractions and acidic polysaccharide fraction, respectively. For each fraction, an ultrafiltration unit of 10,000 MWCO was used to remove small molecules such as monosaccharides in the recovered sample while replacing the solvent with purified water.

As a result, neutral polysaccharide fractions and acidic polysaccharide fractions were isolated and purified as polysaccharides produced by the strain IJH-SONE68.

A semisynthetic medium for producing polysaccharides was prepared by modifying a medium disclosed in Kimmel S A, Roberts R F., “Development of a growth medium suitable for exopolysaccharide production by Lactobacillus delbrueckii ssp. Bulgaricus R R.”, Int. J. Food Microbiol., 40, 87-92 (1998), as follows:
Semisynthetic medium for producing polysaccharides [g/L]
Glucose 20
Tween 80 1.0
Ammonium citrate 2.0
Sodium acetate 5.0
MgSO₄·7H₂O 0.1
MnSO₄·5H₂O 0.05
K₂HPO₄ 2.0
Bacto casitone 10.0
Vitamin Soln. 2 mL
Trace element Soln. 1 mL
Vitamin Soln. [g/L]
4-Aminobenzoic acid 0.05
Biotin 0.001
Folic acid 0.025
Lipoic acid 0.025
Nicotinic acid 0.1
Pantothenic acid 0.05
Pyridoxamin-HC1 0.25
Vitamin B₁₂ 0.05
Pyridoxine 0.025
Riboflavin 0.05
Thiamine 0.1

Trace element Soln. is disclosed in Kets E P W, Galinski E A, de Bont J A M. Carnitine: “A novel compatible solute in Lactobacillus plantarum”, Arch. Microbiol., 192, 243-248 (1994), and the composition is as follows:
Trace element Soln. [g/L]
25% HCl mL
FeCl₂·4H₂O 1.5
CoCl₂·6H₂O 0.19
MnCl₂·4H₂O 0.1
ZnCl₂ 0.07
H₃BO₃ 0.006
Na₂MoO₄·2H₂O 0.036
NiCl₂·6H₂O 0.024
CuCl₂·2H₂O 0.002

Phenol sulfuric acid method (DuBois M, Gilles K A, Hamilton J K, Rebers P A, Smith F., “Colorimetric method for determination of sugars and related substances”, Anal. Chem., 28, 350-356 (1956))

30 μL of a subject sample was mixed with an equal amount of 5 w/v % phenol aqueous solution, and 150 μL of a concentrated sulfuric acid was added to the resultant mixture and mixed with each other to allow a reaction to start. Immediately after 10 minutes, the reaction solution was cooled by ice to stop the reaction. The concentration of saccharides was obtained by measuring the absorbance of the reaction solution at 490 nm. The concentration was determined using a calibration curve prepared by performing the same experiment using glucose as a standard.

### 2. Structural Analysis of Neutral Exopolysaccharide

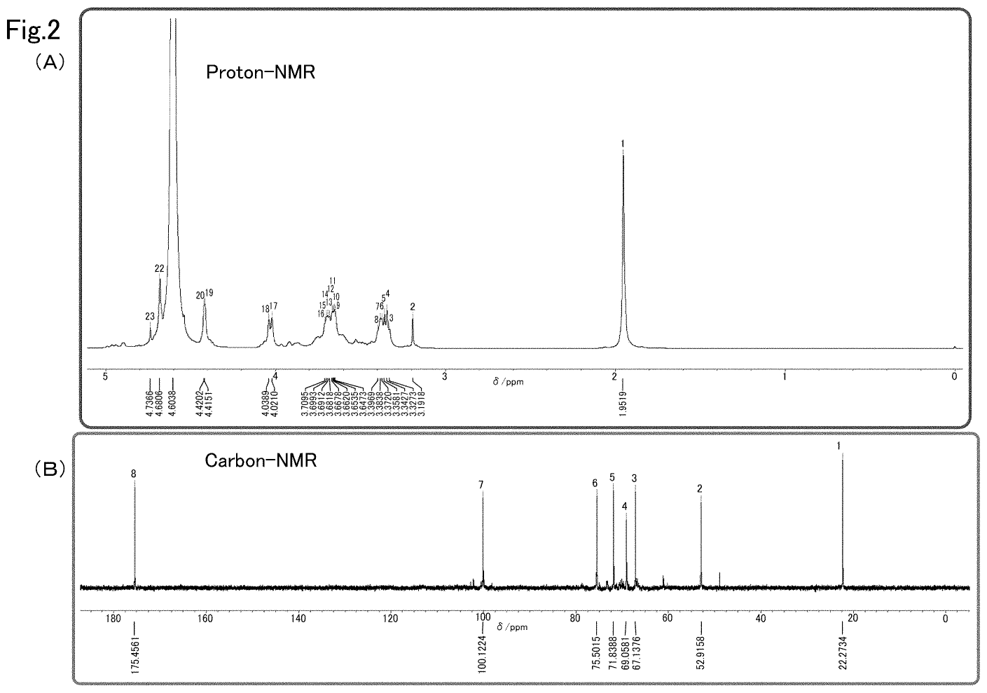

The neutral polysaccharide purified by the aforementioned anion exchange column chromatography (TOYOPEARL DEAE-650 M resin (Tosoh Corporation)) was subjected to proton-NMR and carbon-NMR, and the obtained NMR profiles are each illustrated in FIG. 2. The structural analysis results of the neutral polysaccharide from these NMR profiles are illustrated in FIG. 3.

From the structural analysis results, it was revealed that the neutral exopolysaccharide produced by the strain IJH-SONE68 has a structure in which N-acetylglucosamines are linked with each other via α-1,6 bond.

### 3. Saccharide Composition Analysis of Acidic Exopolysaccharide

The saccharide composition analysis of the aforementioned acidic polysaccharide purified by the anion exchange column chromatography was performed by measuring the composition by a high performance liquid chromatography (HPLC) method.

A 7-fold diluted sample solution was prepared by mixing 10 μL of the purified acidic polysaccharide (7.3 mg/mL) and 60 μL of water and placed in a test tube. 20 μL of the diluted sample solution was collected from the test tube, dried under reduced pressure, and 100 μL of 2 mol/L trifluoroacetic acid was added thereto to dissolve the dried sample. The resultant solution was substituted with nitrogen, sealed under a reduced pressure, hydrolyzed at 100° C. for 6 hours, and then dried under a reduced pressure. To the obtained residue, 200 μL of water was added, dissolved, and filtrated with 0.22 μm filter, to obtain a sample solution for measurement. The sample solution for measurement was 10-fold diluted with water to obtain a sample solution for dilution measurement. 50 μL of each of these sample solutions was analyzed. HPLC system: LC-20A system (Shimadzu Corporation) and spectrofluorophotometer M-10AxL (Shimadzu Corporation) were used as analytical instruments. The analysis conditions were as follows:
Column: TSK-gel Sugar AXG 4.6 mml. D.×15 cm (Tosoh Corporation)
Column temperature: 70° C.
Mobile phase: 0.5 mol/L potassium borate buffer, pH 8.7
Mobile phase flow rate: 0.4 mL/min
Post column labeling: reaction reagent: 1 w/v % arginine·3 w/v % boric acid
Reaction reagent flow rate: 0.5 mL/min
Reaction temperature: 150° C.
Detection wavelength: Ex. 320 nm Em. 430 nm

Chromatograms of samples prepared from the acidic polysaccharide and calibration curve data of each monosaccharide were obtained. The concentrations of constituent saccharides of the acidic polysaccharide in the samples were determined from calibration curves. The obtained results are shown in Table 2.

TABLE 2
Constituent saccharides of acidic exopolysaccharide

| Acidic exopolysaccharide | | Concentration in sample (mg/mL) |
|---|---|---|
| Monosaccharides | Rhamnose | 0.0204 |
| | Ribose | n.d. |
| | Mannose | 3.43 |
| | Arabinose | n.d. |
| | Galactose | 0.0384 |
| | Xylose | n.d. |
| | Glucose | 0.219 |

In the Table, n.d. indicates no detection.

### Example 3

Inhibition activity of the strain IJH-SONE68 and polysaccharides thereof against production of inflammatory cytokines in cultured cells

The cultured liquid of the strain IJH-SONE68 obtained in Example 1, and the polysaccharide sample containing the neutral polysaccharide fractions and the acidic polysaccharide fractions, which were polysaccharides produced by the strain IJH-SONE68 and obtained in Example 2, were studied for an inhibition activity against production of inflammatory cytokines in cultured cells, according to the method disclosed below.

### 1. Test Methods

A glycerol stock of the strain IJH-SONE68 obtained in Example 1, which had been stored in a freezer at −80° C., was inoculated into MRS broth so that the final concentration was 1 v/v %, and statically cultured at 28° C. for 24 hours. Subsequently, this seed culture liquid was inoculated into MRS broth so that the concentration was 1 v/v %, and statically cultured at 28° C. for 24 hours. The resultant culture liquid supernatant was sterilized by a filter, pulverized by a freeze-dryer, and then suspended in DMEM medium having the same volume as the volume of the culture liquid supernatant before freeze-dried. The thus obtained suspension was used as a culture liquid supernatant sample.

Human colon cancer-derived Caco-2 cells were used as intestinal epithelial cell models. The Caco-2 cells were cultured in DMEM medium containing 10 (v/v) % fetal bovine serum (FBS) at 37° C. under 5% CO₂ conditions until the cells became 70% confluent. Then, after the resultant cells were incubated for 30 minutes in DMEM medium containing 5 (v/v) % of the above-mentioned culture liquid supernatant sample or the polysaccharide (EPS) sample of the strain IJH-SONE68 obtained in Example 2, inflammation was caused in the Caco-2 cells by pathogenic bacteria, Salmonella and Campylobacter, and the amount of an inflammatory cytokine, interleukin-8 (IL-8), secreted from the Caco-2 cells was measured 24 hours later.

Similarly, mouse macrophage cultured cells RAW264.7 were cultured in DMEM medium containing 10 (v/v) % FBS. Then, the resultant cultured cells were incubated in DMEM medium containing 5 (v/v) % of the above-mentioned culture liquid supernatant sample for 30 minutes, and inflammation was caused by 2 μg/mL E. coli 0111 strain-derived lipopolysaccharide (LPS). The amount of secreted inflammatory cytokines, tumor necrosis factor-α (TNF-α) and interleukin-6 (IL-6), were measured 24 hours later.

### 2. Test Results

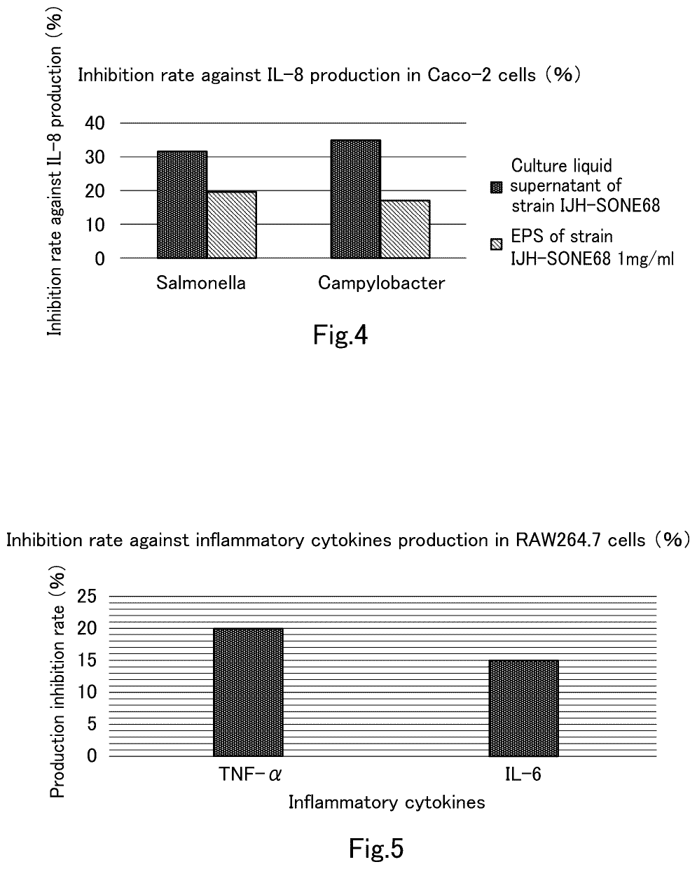

FIG. 4 shows results of measuring the inhibition rates of the culture liquid supernatant of the strain IJH-SONE68 and the polysaccharides against IL-8 production in the Caco-2 cells. FIG. 5 shows results of measuring the inhibition rate of the culture liquid supernatant of the strain IJH-SONE68 against the production of TNF-a and IL-6 in mouse macrophage cultured cells RAW264.7.

As shown in FIG. 4, the culture liquid supernatant of the strain IJH-SONE68 exhibited an inhibition activity on IL-8 production against Salmonella and Campylobacter. In addition, while the inhibition activity of the crudely purified EPS on IL-8 production was low as compared to that of the culture liquid supernatant of the strain IJH-SONE68, the crudely purified EPS exhibited an inhibition activity on IL-8 production. On the other hand, as shown in FIG. 5, the culture liquid supernatant of the strain IJH-SONE68 exhibited inhibition activity rates of 19.9% and 15% on productions of inflammatory cytokines, TNF-a and IL-6, in the RAW264.7 macrophage, respectively.

### Example 4

### 1. Improvement Effect of Polysaccharides of Strain IJH-SONE68 on Ulcerative Colitis Model Mice

The improvement effect of the polysaccharides produced by the strain IJH-SONE68 obtained in Example 2 on ulcerative colitis model mice were studied according to methods disclosed below.

### (1) Test Methods

In the present test, C57BL/6J Jms Slc (SPF) male mice were used. Seven-week-old mice were brought in, and three mice were kept in each breeding cage. After one week of acclimatization using a normal feed (MF, manufactured by Oriental Yeast Co., Ltd.), the mice were divided into groups, each of which consisted of three mice (Groups A to D disclosed below), and ingestion of the polysaccharide (EPS) samples produced by the strain IJH-SONE68 obtained in Example 2 was started. At this time, in addition to the EPS samples produced by the strain IJH-SONE68, a group, in which EPS purified from other EPS-producing lactic acid bacterium strain Pediococcus pentosaceus LP28 (PLoS One.7: e30696 (2012); Eur. J. Clin. Nutr. 70:582-587 (2016)) was ingested, was provided as a comparative control.

After one-week EPS ingestion period, the drinking water was changed to 3 w/v % aqueous solution of sodium dextran sulfate (DSS), and induction of ulcerative colitis was started. During this period, the EPS ingestion was continued, and both normal feed and drinking water were allowed to be ingested freely. Ingestion of EPS was made as an imperative oral ingestion by sonde, and 200 μL of EPS sample was ingested per mouse per day.
Group A: a group in which ulcerative colitis was not induced (ingestion of sterile distilled water, instead of EPS) (Negative Control Group (NC));
Group B: a group in which sterile distilled water was ingested, instead of EPS (3 w/v % DSS was used as drinking water) (Positive Control Group (PC));
Group C: a group in which the EPS fractions crudely purified from the strain IJH-SONE68 were ingested (3 w/v % DSS was used as drinking water. The preparation was made by dissolving the freeze-dried product of the EPS fractions in sterile distilled water at a concentration of 5 mg/mL);
Group D: a group in which the EPS fractions crudely purified from Pediococcus pentosaceus LP28 were ingested (3 w/v % DSS was used as drinking water. The preparation was made by dissolving the freeze-dried product of the EPS fractions in sterile distilled water at a concentration of 5 mg/mL).

After the induction of ulcerative colitis was started, the breeding was continued for another week. The diarrhea score and bloody stools score shown below were recorded after the start of induction.

Diarrhea score:
0 points: normal stools;
1 point: slightly soft stools (can be collected with tweezers);
2 points: fairly soft stools (cannot be collected with tweezers);
3 points: watery stools

Bloody stools score:
0 points: brown (normal stools);
1 point: bloody stools colored red only on the surface;
2 points: bloody stools colored red up to the inside;
3 points: from reddish brown stools to black stools.

In addition, after the one-week induction period was completed, the mice were euthanized by isoflurane inhalation anesthesia, the large intestine was collected, and the length was measured.

Tukey-Kramer method was used as a significance test for each measurement data, and when the risk factor rate (p-value) was less than 0.05, it was judged to be “significantly different”.

### (2) Test Results

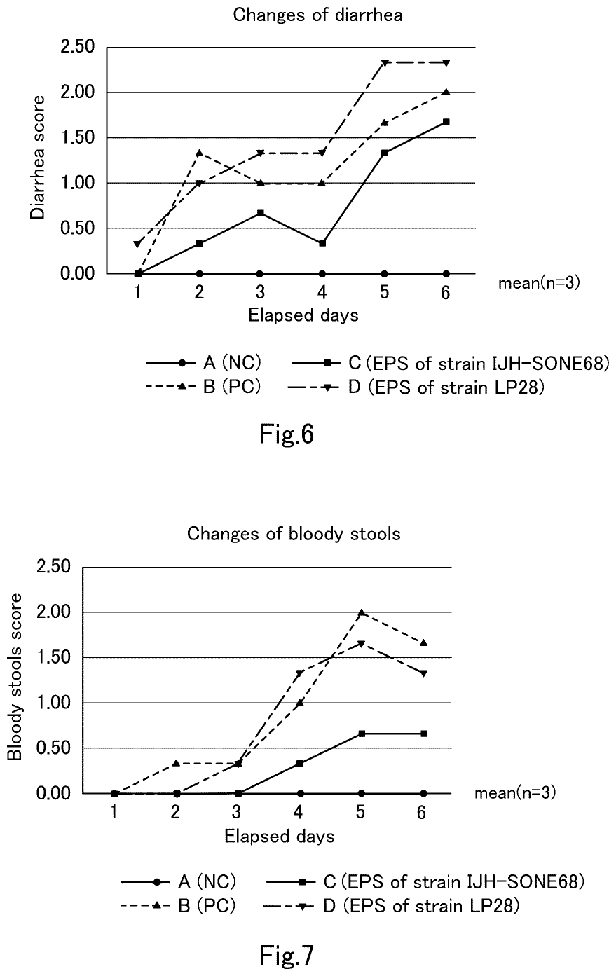

a) FIG. 6 shows the changes in the diarrhea score (mean value) from the start date of DSS ingestion in each group. As can be seen from FIG. 6, no diarrhea was observed during the period in the negative control group (Group A) in which DSS was not ingested and in which ulcerative colitis was not developed. In the other three groups (Groups B to D) in which DSS was ingested, including the positive control group (Group B) in which the EPS sample was not ingested, the softening of stools progressed with each passing day, that is, it was recognized that the diarrhea score increased. However, in the group (Group C) in which the EPS of the strain IJH-SONE68 was ingested, the progression of the softening stools was slow, and the progression of the softening stools was suppressed as compared to the group (Group D) in which the EPS of Pediococcus pentosaceus LP28 was ingested.

b) FIG. 7 shows the changes in the bloody stools score (mean value) from the start date of DSS ingestion in each group. No bloody stools were observed during the period in the negative control group (Group A) in which DSS was not ingested and in which ulcerative colitis was not developed. In the other three groups (Groups B to D) in which DSS was ingested, including the positive control group (Group B) in which the EPS sample was not ingested, the worsening of bloody stools symptom was observed day by day, and it was recognized that the bloody stools score increased. However, in the group (Group C) in which the EPS of the strain IJH-SONE68 was ingested, the worsening progression of bloody stools was clearly slow, and the worsening progression of bloody stools was suppressed as compared to the group (Group D) in which the EPS of Pediococcus pentosaceus LP28 was ingested.

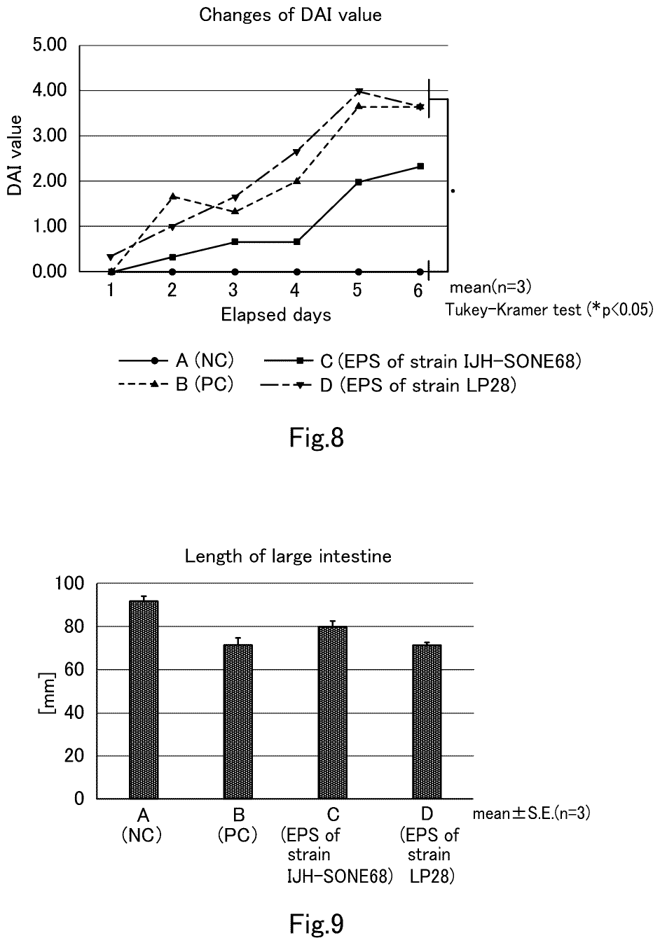

c) The total value of the diarrhea score and the bloody stools score was defined as DAI (Disease activity index), and FIG. 8 shows the results of comparing the changes in the DAI value of each group. As can be seen from FIG. 8, as compared to the negative control group (Group A) in which no increase in the DAI value was observed, in the other three groups (Groups B to D) in which DSS was ingested, including the negative control group (Group B) in which the EPS sample was not ingested, daily increases in the DAI value were observed. On the final day of observation, the DAI value of the positive control group (Group B) changed similarly to that of the group (Group D) in which the EPS of Pediococcus pentosaceus LP28 was ingested and was significantly higher than that of the negative control group (Group A). On the other hand, in the group (Group C) in which the EPS of the strain IJH-SONE68 was ingested, the increase in the DAI value was only about half and significantly suppressed. That is, no influence on the DAI value was recognized in the group (Group D) in which the EPS of the other strain was ingested, but the effect of suppressing the increase in the DAI value was recognized only in the group (Group C) in which the EPS of the strain IJH-SONE68 was ingested.

d) After the test period passed, the large intestine was removed from the individuals in each group, and FIG. 9 shows the results of comparing the lengths of the large intestines, which are considered to shorten with inflammation. As can be seen from FIG. 9, the large intestinal lengths of the positive control group (Group B) and the group (Group D) in which the EPS of Pediococcus pentosaceus LP28 was ingested were almost the same as each other, and the shortening of about 20 cm was observed as compared to those of the negative control group (Group A). On the other hand, in the group in which the EPS of strain IJH-SONE68 was ingested (Group C), although no significant difference was recognized, the shortening was only about 10 cm, and it was observed that inflammation was suppressed.

### 2. Improvement Effect of the Neutral and Acidic Polysaccharides of the Strain IJH-SONE68 on Ulcerative Colitis Model Mouse

The improvement effect of the neutral and acidic polysaccharides produced by the strain IJH-SONE68 obtained in Example 2 on ulcerative colitis model mice was studied according to methods disclosed below.

### (1) Test Methods

In the present test, C57BL/6J Jms Slc (SPF) male mice were used. Seven-week-old mice were brought in, and five mice were kept in each breeding cage. After one week of acclimatization using a normal feed (MF, manufactured by Oriental Yeast Co., Ltd.), the mice were divided into groups, each of which consisted of five mice (Groups A to D disclosed below), and ingestion of the neutral polysaccharide (EPS) and acidic polysaccharide (EPS) samples produced by the strain IJH-SONE68 obtained in Example 2 was started. After one-week EPS ingestion period, the drinking water was changed to 3 w/v % aqueous solution of sodium dextran sulfate (DSS), and induction of ulcerative colitis was started. During this period, the EPS ingestion was continued, and both normal feed and drinking water were allowed to be ingested freely. Ingestion of EPS was made as an imperative oral ingestion by sonde, and 200 μL of EPS sample was ingested per mouse per day.
Group A: a group in which ulcerative colitis was not induced (ingestion of sterile distilled water, instead of EPS) (Negative Control Group (NC));
Group B: a group in which sterile distilled water was ingested, instead of EPS (3 w/v % DSS was used as drinking water) (Positive Control Group (PC));
Group C: a group in which the neutral EPS fractions crudely purified from the strain IJH-SONE68 were ingested (3 w/v % DSS was used as drinking water. The preparation was made by dissolving the freeze-dried product of the neutral EPS fractions in sterile distilled water at a concentration of 1 mg/mL);
Group D: a group in which the acidic EPS fractions crudely purified from the strain IJH-SONE68 (3 w/v % DSS was used as drinking water. The preparation was made by dissolving the freeze-dried product of the acidic polysaccharide fractions in sterile distilled water at a concentration of 1 mg/mL).

After the induction of ulcerative colitis was started, the breeding was continued for another week. The diarrhea score and bloody stools score shown below were recorded after the start of induction.

Diarrhea score:
0 points: normal stools;
1 point: slightly soft stools (can be collected with tweezers);
2 points: fairly soft stools (cannot be collected with tweezers);
3 points: watery stools

Bloody stools score:
0 points: brown (normal stools);
1 point: bloody stools colored red only on the surface;
2 points: bloody stools colored red up to the inside;
3 points: from reddish brown stools to black stools.

In addition, after the one-week induction period was completed, the mice were euthanized by isoflurane inhalation anesthesia, the large intestine was collected, and the length was measured.

Furthermore, proteins were extracted from the collected large intestine, and myeloperoxidase (MPO) activity, which is an index of inflammation intensity, was measured and compared with that of each group. Specifically, first, the large intestine fragment which had been cut to a length of several mm was washed with 50 mM potassium phosphate buffer (pH 6.0), a buffer solution containing 0.5 w/v % hexadecyltrimethylammonium bromide was then added to the large intestine fragment at an amount of 40 μL per 1 mg of the tissue, and the large intestine fragment was homogenized using a homogenizer pestle. After homogenization, the resultant solution, from which the insoluble fraction had been removed by centrifugation, was used as an enzyme solution.

A reaction solution was prepared by adding o-dianisidine dihydrochloride and hydrogen peroxide to 5 mM potassium phosphate buffer (pH 6.0) so that the concentrations of o-dianisidine dihydrochloride and hydrogen peroxide were 0.53 mM and 44 μM, respectively. 200 μL of this reaction solution and 7 μL of the enzyme solution were mixed, and the change in absorbance at 450 nm over 3 minutes was measured. At this time, unit in which 1 μmol of hydrogen peroxide was decomposed per minute was defined as one unit.

Tukey-Kramer method was used as a significance test, and when the risk factor rate (p value) was less than 0.05, it was judged to be “significantly different”.

### (2) Test Results

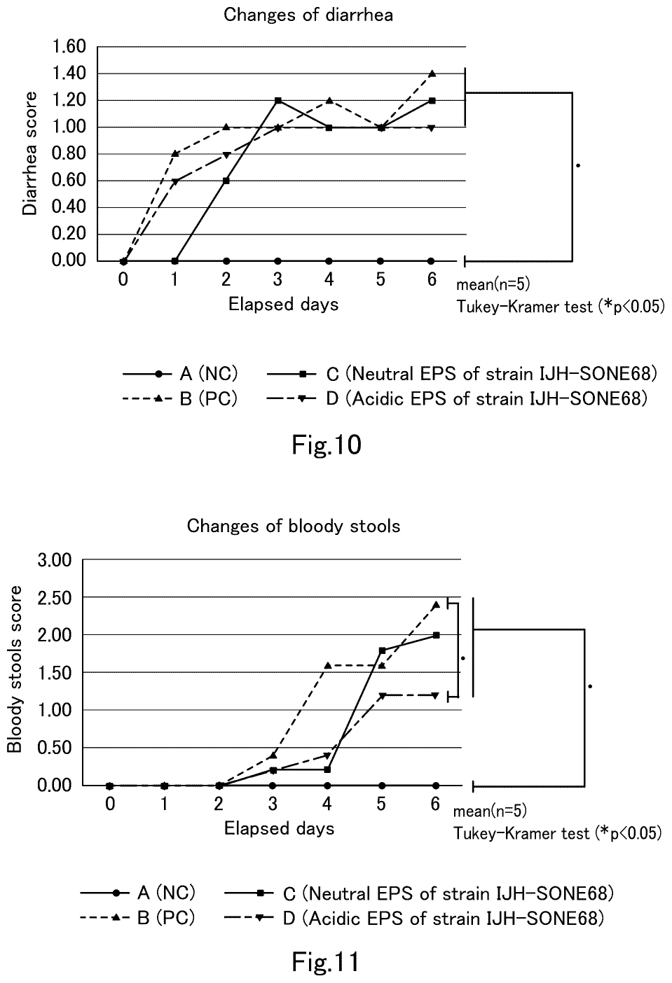

a) FIG. 10 shows the changes in the diarrhea score (mean value) from the start date of DSS ingestion in each group. As can be seen from FIG. 10, no diarrhea was observed during the period in the negative control group (Group A) in which DSS was not ingested and in which ulcerative colitis was not developed. In the other three groups (Groups B to D) in which DSS was ingested, including the positive control group (Group B) in which the EPS sample was not ingested, the softening of stools progressed with each passing day, that is, it was recognized that the diarrhea score increased.

b) FIG. 11 shows the changes in the bloody stools score (mean value) from the start date of DSS ingestion in each group. As can be seen from FIG. 11, no bloody stools were observed during the period in the negative control group (Group A) in which DSS was not ingested and in which ulcerative colitis was not developed. In the other three groups (Groups B to D) in which DSS was ingested, including the positive control group (Group B) in which the EPS sample was not ingested, the worsening of bloody stools symptom was observed day by day, and it was recognized that the bloody stools score increased. However, in the group (Group D) in which the acidic EPS of the strain IJH-SONE68 was ingested, the bloody stools score was significantly suppressed as compared to the positive control group (Group B).

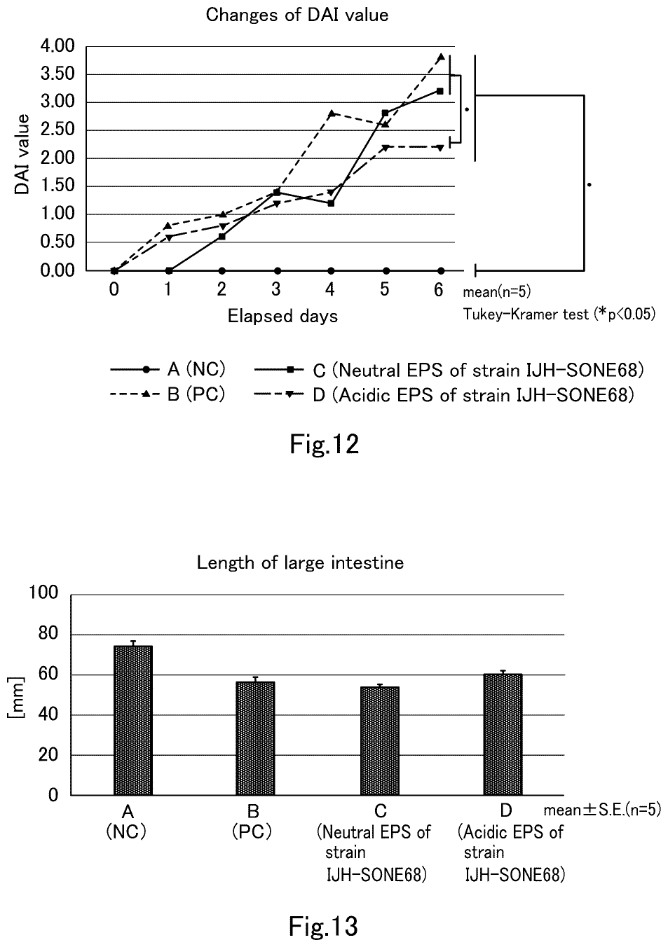

c) The total value of the diarrhea score and the bloody stools score was defined as DAI (Disease activity index), and FIG. 12 shows the results of comparing the changes in the DAI value of each group. As can be seen from FIG. 12, as compared to the negative control group (Group A) in which no increase in the DAI value was observed, daily increases in the DAI value were observed in the other three groups (Groups B to D) in which DSS were ingested, including the negative control group (Group B) in which the EPS sample was not ingested. On the final day of observation, the DAI value of each group (Groups B to D) was significantly higher than that of the negative control group (Group A). However, in the group (Group D) in which the acidic EPS of the strain IJH-SONE68 was ingested, the increase in the DAI value was only about half of that of the positive control (Group B), and the increase was significantly suppressed. That is, it was suggested that the acidic EPS of the strain IJH-SONE68 is stronger than the neutral EPS of the strain IJH-SONE68 in terms of the action of improving diarrhea and bloody stools of the ulcerative colitis model mice.

d) After the test period passed, the large intestine was removed from the individuals in each group, and FIG. 13 shows the results of comparing the lengths of the large intestine, which are considered to shorten with inflammation. As can be seen from FIG. 13, the large intestinal lengths of the positive control group (Group B) and the group (Group C) in which the neutral EPS of the strain IJH-SONE68 was ingested were almost the same as each other, and the shortening of about 20 cm was observed as compared to that of the negative control group (Group A). On the other hand, in the group (Group D) in which the acidic EPS of strain IJH-SONE68 was ingested, although no significant difference was recognized, the shortening was only about 15 cm, and it was suggested from the present results that the acidic EPS of the strain IJH-SONE68 more suppresses inflammation.

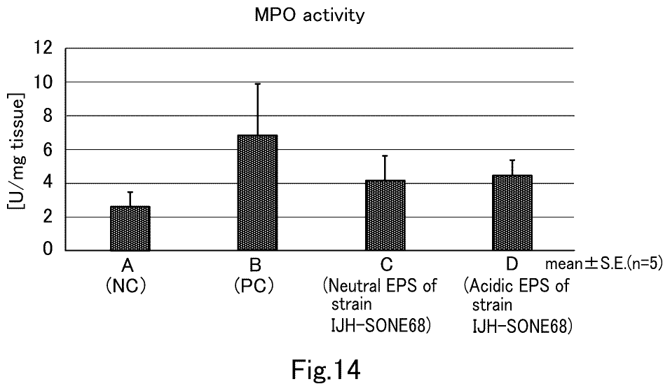

e) Proteins were extracted from the large intestine of each individual in each group, the MPO activities in each group, which are considered to increase with inflammation, were compared, and the obtained results are shown in FIG. 14. As can be seen from FIG. 14, the MPO activity in the positive control group (Group B) apparently increased as compared to that in the negative control (Group A), but the increase was suppressed in the group (Group C) in which the neutral EPS of the strain IJH-SONE68 was ingested and the group (Group D) in which the acidic EPS of the strain IJH-SONE68 was ingested. In this activity, there was no large difference in the effect between the neutral and acidic EPSs of the strain IJH-SONE68.

As is clear from the foregoing, it has been revealed that when the culture liquid supernatant of Lactobacillus paracasei IJH-SONE68 or polysaccharides thereof is added to human colon cancer-derived Caco-2 cells, which are intestinal epithelial model cells and in which inflammation has been induced by Salmonella and Campylobacter, the expression and secretion of interleukin-8 (IL-8), which is one of inflammatory cytokines, are suppressed. In addition, it has been revealed that when the culture liquid supernatant of Lactobacillus paracasei IJH-SONE68 is added to cultured mouse macrophage cells RAW264.7 in which inflammation has been induced by lipopolysaccharide, the expression and secretion of tumor necrosis factor-α (TNF-α) and interleukin-6 (IL-6), which are inflammatory cytokines, are suppressed.

Furthermore, it has been revealed that when polysaccharides, neutral polysaccharides, acidic polysaccharides, or the like of Lactobacillus paracasei IJH-SONE68 are orally administered to ulcerative colitis model mice which have been developed by ingestion of sodium dextran sulfate (DSS), diarrhea and bloody stools are suppressed, and myeloperoxidase (MPO) activity in the large intestine, which is an index of inflammation intensity, is suppressed, whereby the worsening of the disease condition is suppressed to improve the disease condition.

As is clear from the foregoing detailed disclosures, the present invention provides the following inventions:

[1] A composition for improving, preventing or treating an inflammatory bowel disease, comprising, as an active ingredient, a cell body of a fig-derived lactic acid bacterium belonging to Lactobacillus paracasei, a cultured or fermented product thereof, or a polysaccharide produced thereby.

[2] The composition according to the above [1], wherein the inflammatory bowel disease is ulcerative colitis or Crohn's disease.

[3] The composition according to the above [1] or [2], wherein the lactic acid bacterium is Lactobacillus paracasei strain IJH-SONE68 (Accession No. NITE BP-02242) or a lactic acid bacterium equivalent thereto.

[4] The composition according to any one of the above [1] to [3], wherein the polysaccharide is a neutral polysaccharide or an acidic polysaccharide.

[5] The composition according to the above [4], wherein the acidic polysaccharide is an acidic polysaccharide composed mainly of glucoses and mannoses.

[6] The composition according to the above [4], wherein the neutral polysaccharide is a neutral polysaccharide having a structure in which N-acetylglucosamines are linked with each other via α-1,6 bond.

[7] The composition according to any one of the above [1] to [6], wherein the composition is a food or drink composition.

[8] The composition according to the above [7], wherein the food or drink is a beverage, a functional food, a fermented food, or a supplement.

[9] The composition according to any one of the above [1] to [6], wherein the composition is a pharmaceutical composition.

[10] The composition according to any one of the above [1] to [6], wherein the composition is a feed composition.

[11] Use of a cell body of a fig-derived lactic acid bacterium belonging to Lactobacillus paracasei, a cultured or fermented product thereof, or a polysaccharide produced thereby, as an active ingredient of a composition for improving, preventing or treating an inflammatory bowel disease.

[12] The use according to the above [11], wherein the inflammatory bowel disease is ulcerative colitis or Crohn's disease.

[13] The use according to the above [11] or [12], wherein the lactic acid bacterium is Lactobacillus paracasei strain IJH-SONE68 (Accession No. NITE BP-02242) or a lactic acid bacterium equivalent thereto.

[14] The use according to any one of the above to [13], wherein the polysaccharide is a neutral polysaccharide or an acidic polysaccharide.

[15] The use according to the above [14], wherein the acidic polysaccharide is an acidic polysaccharide composed mainly of glucoses and mannoses.

[16] The use according to the above [14], wherein the neutral polysaccharide is a neutral polysaccharide having a structure in which N-acetylglucosamines are linked with each other via α-1,6 bond.

[17] The use according to any one of the above [11] to [16], wherein the composition is a food or drink composition.

[18] The use according to the above [17], wherein the food or drink is a beverage, a functional food, a fermented food, or a supplement.

[19] The use according to any one of the above [11] to [16], wherein the composition is a pharmaceutical composition.

[20] The use according to any one of the above [11] to [16], wherein the composition is a feed composition.

[21] A method of improving, preventing or treating an inflammatory bowel disease in a subject, which comprises applying to the subject in need thereof a composition comprising, as an active ingredient, a cell body of a fig-derived lactic acid bacterium belonging to Lactobacillus paracasei, a cultured or fermented product thereof, or a polysaccharide produced thereby.

[22] The method according to the above [21], wherein the inflammatory bowel disease is ulcerative colitis or Crohn's disease.

[23] The method according to the above [21] or [22], wherein the lactic acid bacterium is Lactobacillus paracasei strain IJH-SONE68 (Accession No. NITE BP-02242) or a lactic acid bacterium equivalent thereto.

[24] The method according to any one of the above [21] to [23], wherein the polysaccharide is a neutral polysaccharide or an acidic polysaccharide.

[25] The method according to the above [24], wherein the acidic polysaccharide is an acidic polysaccharide composed mainly of glucoses and mannoses.

[26] The method according to the above [24], wherein the neutral polysaccharide is a neutral polysaccharide having a structure in which N-acetylglucosamines are linked with each other via α-1,6 bond.

[27] The method according to any one of the above [21] to [26], wherein the composition is a food or drink composition.

[28] The method according to the above [27], wherein the food or drink is a beverage, a functional food, a fermented food, or a supplement.

[29] The method according to any one of the above [21] to [26], wherein the composition is a pharmaceutical composition.

[30] The method according to any one of the above [21] to [26], wherein the composition is a feed composition.

### INDUSTRIAL APPLICABILITY

As disclosed in detail herein above, the composition of the present invention is effective in improving, preventing or treating an inflammatory bowel disease and can be particularly used to improve, prevent or treat ulcerative colitis and Crohn's disease. The composition of the present invention is preferably used as a food or drink, a medicament, or a feed for improving, preventing or treating an inflammatory bowel disease. Since the composition of the present invention comprises, as an active ingredient, a cell body of a fig-derived lactic acid bacterium belonging to Lactobacillus paracasei, a cultured or fermented product thereof, or a polysaccharide produced thereby, the composition of the present invention has high safety, can be applied for a long period of time, can be supplied in a large amount at low cost, and has extremely high utility and practicality.

1-10. (canceled)

11. A method for improving, preventing or treating an inflammatory bowel disease, the method comprising: administering a composition comprising a cell body of Lactobacillus paracasei strain IJH-SONE68 (Accession No. NITE BP-02242), a cultured or fermented product thereof, or a polysaccharide thereof to a subject in need thereof.

12. The method according to claim 11, wherein the inflammatory bowel disease is ulcerative colitis.

13. The method according to claim 11, wherein the inflammatory bowel disease is Crohn's disease.

14. The method according to claim 11, wherein the polysaccharide is a neutral polysaccharide.

15. The method according to claim 11, wherein the polysaccharide is an acidic polysaccharide.

16. The method according to claim 14, wherein the neutral polysaccharide is a neutral polysaccharide having a structure in which N-acetylglucosamines are linked with each other via α-1,6 bond.

17. The method according to claim 15, wherein the acidic polysaccharide is an acidic polysaccharide composed of glucoses, mannoses, galactoses, and rhamnoses.

18. The method according to claim 11, wherein the composition is a food composition.

19. The method according to claim 11, wherein the composition is a drink composition.

20. The method according to claim 18, wherein the food composition is a beverage, a functional food, a fermented food, or a supplement.

21. The method according to claim 19, wherein the drink composition is a beverage, a functional food, a fermented food, or a supplement.

22. The method according to claim 11, wherein the composition is a pharmaceutical composition.

23. The method according to claim 11, wherein the composition is a feed composition.
